(19) United States
(12) Patent Application Publication
(10) Pub. No.: US 2008/0097387 A1
(43) Pub. Date: Apr. 24, 2008

(54) ORAL CAVITY LIQUID DELIVERY SYSTEM INCLUDING PRE-ANGLED NEEDLE ASSEMBLY AND METHOD FOR USING THE SAME

(76) Inventor: David M. Spector, Roseville, CA (US)

Correspondence Address: CRAIG A. SIMMERMON, 251 LYMAN CIR., SACRAMENTO, CA, 95835, US

(21) Appl. No.: 11/309,645

(22) Filed: Sep. 4, 2006

(51) Int. Cl. A61M 5/28

(52) U.S. Cl. 604/512; 604/192; 604/200; 604/77; 433/216

(57) ABSTRACT

A delivery system for dispensing sterile liquids into the oral cavity using a novel pre-angled needle hub assembly attached to a standard anesthetic syringe loaded with a novel cartridge ampule containing a certain liquid solution. This system substantially improves the efficiency of delivering small amounts of sterile fluid into the oral cavity by hypodermic means or by irrigation means using pre-angled needles or cannulas to reach otherwise impossible-to-reach locations within the oral cavity while also providing the ability to dispose of used needles without re-bending and thus complying with federal OSHA, state OSHA, and state regulatory disposal regulations.

### BACKGROUND OF INVENTION

Devices currently available to dental practitioners used to deliver small amounts, less than two cubic centimeters, of sterile irrigation liquid or anesthetic solution into the oral cavity are prone to problems related to needle disposal safety; effective, efficient, and ergonomic use by the practitioner; high cost; timely set-up requirements; and other issues. This invention combines a novel needle/hub assembly, a novel cartridge ampule, and a standard anesthetic syringe or standard intraligamental syringe to yield an oral cavity liquid delivery system that is surprisingly simple, extremely effective, easy to use, inexpensive, and capable of delivering liquid to oral cavity locations that are currently impossible to do so without resorting to illegal needle bending and disposal methods. Specifically, this invention utilizes a “pre-bent” needle or cannula in conjunction with a cartridge ampule containing sterile fluid which allows the practitioner to efficiently, effectively, and legally complete many procedures such as intrapulpal injection (depicted in FIG. 8), intraligamental injection (PDL) technique (depicted in FIG. 9), anesthetizing accessory nerves on the lingual aspect of the lower posterior teeth, various endodontic sterile irrigation procedures, and sterile irrigation procedures conducted during oral various surgical procedures, including, but not limited to, tooth extraction, periodontal surgery, endodontic surgery, biopsy procedures, implant surgery, and restorative dental procedures.

With anesthetic delivery, certain procedures physically require the use of angled needles to reach certain locations in the oral cavity. As there are no pre-bent needles available to the practitioner through current supply channels, the practitioner must bend a straight needle himself. In this instance, the practitioner or his employee must then re-bend the used needle in order to re-cap the needle before disposal or, even worse, simply dispose the needle in a bent and uncapped condition. However, federal and state OSHA regulations commonly call for needles to be capped before disposal and for needles not to be bent or broken for the purpose of disposal. Therefore, whether the practitioner re-bends to re-cap or disposes without re-capping, the practitioner is not in compliance with federal and state OSHA regulations when he bends a needle to deliver anesthetic or for any other purpose.

The current invention includes a pre-bent needle assembly, in a variety of angles, that is attached to a conventional anesthetic syringe. The practitioner simply attaches the appropriate needle assembly to the syringe, completes the anesthetic delivery procedure, re-caps the needle assembly, and disposes the needle. As with standard straight needle disposal, disposal of needles of the current invention does not require re-bending in order re-cap, finally allowing practitioners to dispose of angled needles legally and properly.

The needle assembly element of this invention is somewhat similar to U.S. Pat. No. 5,514,113 (Anderson); however, Anderson claims a different and less useful needle hub angle range. Anderson claims an angle ranging between twenty-five and forty-five degrees while this invention includes a best mode embodiment angle of ninety degrees for the corresponding element of the somewhat similar invention. Furthermore, Anderson is not available to practitioners as it is not offered for sale. In any event, the result of the current situation is for practitioners to somehow bend needles to perform certain procedures and then re-bend needles to dispose them. This practice is illegal and unwise.

In addition to incorporating an angle as its best mode that does not read on Anderson, this invention includes other distinguishing characteristics, such as, the ability to perform various sterile oral cavity irrigation procedures with unprecedented ease and overall efficiency than any other procedure for same currently in the public domain. Anderson is useful for injection procedures only while this invention is useful for both irrigation and injection. In fact, its primary usefulness is the ability to perform both of these procedures consecutively with the same instrument, with unprecedented ease and overall efficiency. Additionally, Anderson does not include a mode with needle safety caps. Another substantial benefit of this invention is that it includes a novel device and procedure to allow practitioners to finally dispose of needles safely and legally after performing procedures that require the use of pre-bent needle or cannulas. Other objective indicia of unobviousness of the current invention include substantially probable commercial success, long felt but unresolved needs, failure of others, and recognition of a problem.

The most common small-volume oral cavity irrigation procedure used by dental practitioners is the manual method. Whether the practitioner starts with straight or pre-bent cannulas, this irrigation procedure is prone to problems because of complex and costly set-up requirements. Set-up consists of assembling a large bottle of sterile liquid, a sterile scissor to open the hermetically sealed bottle, a sterile plastic syringe, a sterile cannula, and a sterile vessel in which to pour the sterile liquid so that it may be extracted into the sterile syringe. Manual irrigation methods involve pouring solutions out of a large bottle into a small cup or container placed on a countertop or bracket table from which the practitioner fills and re-fills the syringe. This procedure is unnecessarily time consuming and costly as compared to the procedure to use the current invention.

Additionally, many irrigants are caustic, as with sodium hypochlorite, chloroform, EDTA, hydrogen peroxide, and ethyl alcohol, which can be spilled or splashed onto the patient, dentist, staff, work surface, or equipment which can cause substantial human injury, costly equipment damage, and/or costly clean-up procedures. The proposed invention would eliminate the occurrence of all of these circumstances.

Another common sterile irrigation system is a counter-top bag and tube apparatus, such as model NWS-9 sold by Aseptico. This type of system is even more costly and more cumbersome than the manual system. Hence, the current invention is an improvement over this method as well.

Unfortunately, another very common method of delivering irrigants to the oral cavity actually introduces “un-sterile” irrigants into the patent's system. Many practitioners are using the air/water syringe attached to the dental chair unit to perform irrigation procedures. Typically, practitioners maintain and clean the internal plumbing, i.e. water reservoir, tubing, and hand piece, of the dental unit by flushing these items with water at the beginning of each day and in-between patients. Practitioners do not autoclave dental unit components which come in contact with irrigation solutions. This is unfortunate because dental units are known to become contaminated with “biofilm” and live bacteria after only short periods of use and such contaminants are not eliminated by standard water flushing. Therefore, practitioners are commonly creating risks of infection during irrigation procedures by using non-sterile irrigation water from their dental units.

Pre-bent irrigation cannulas are available to the practitioner according U.S. Pat. No. 6,079,979 (Riitano); however, Riitano differs substantially from and is inferior to the proposed invention. Firstly, Riitano is only attachable to conventional syringes of Luer Lock design. Luer Lock syringes are cumbersome to use as compared to the anesthetic syringe which provides substantially better ergonomics, yielding much more effective control and ease of use, because of its heavier weight and “feel” for which dentists generally have become accustomed. This invention includes an anesthetic syringe while Riitano cannot be attached to this type of syringe. Secondly, Riitano cannot accept cartridge ampules. One of the significant problems in the field of dentistry that this invention solves is the inability of current systems to allow for quick, inexpensive, efficient, and easy delivery of small volumes of sterile irrigation liquid into the oral cavity. The plunger type tubular cartridge element of this invention is the primary facilitating element of this feature; while Riitano cannot accept any type of cartridge ampule. This is another significant distinction. Specifically, Riitano cannot accept cartridge ampules because it lacks a proximal end needle as well as other limitations. Additionally, Riitano is used only for irrigation and cannot be used where a sharp penetrating needle is required to deliver an injection.

Thirdly, Riitano (as well as Anderson) does not claim the use of safety needle covers which greatly reduce the risk of puncture wounds to the dentist and his staff. Needle puncture wounds create a risk of spreading infectious disease and also require the dentist to initiate a very complex and costly regulatory procedure designed to limit the risks created by such accidental needle stick incidents. The current invention would substantially reduce the risk of occurrences of such incidents.

### BRIEF DESCRIPTION OF THE DRAWINGS

Typical embodiments of the present invention are illustrated in the accompanying drawings which are not to be limiting of the invention's scope in which:

FIG. 1 is a side view of pre-angled needle assembly and is the preferred embodiment of pre-angled needle assembly.

FIG. 2 is an end view of the distal end of pre-angled needle assembly.

FIG. 3 is an end view of the proximal end of pre-angled needle assembly.

FIG. 4 is a side view of oral cavity liquid delivery system including pre-angled needle assembly.

FIG. 5 is a perspective view of cartridge ampule with faceted distinguishability means.

FIG. 6 is a bottom end view of cartridge ampule with faceted distinguishability means.

FIG. 7 is a top end view of cartridge ampule with faceted distinguishability means.

FIG. 8 is a depiction of an intrapulpal injection procedure or irrigation procedure using the preferred embodiment of oral cavity liquid delivery system including pre-angled needle assembly.

FIG. 9 is a depiction of an intraligamental injection procedure using the preferred embodiment of oral cavity liquid delivery system including pre-angled needle assembly.

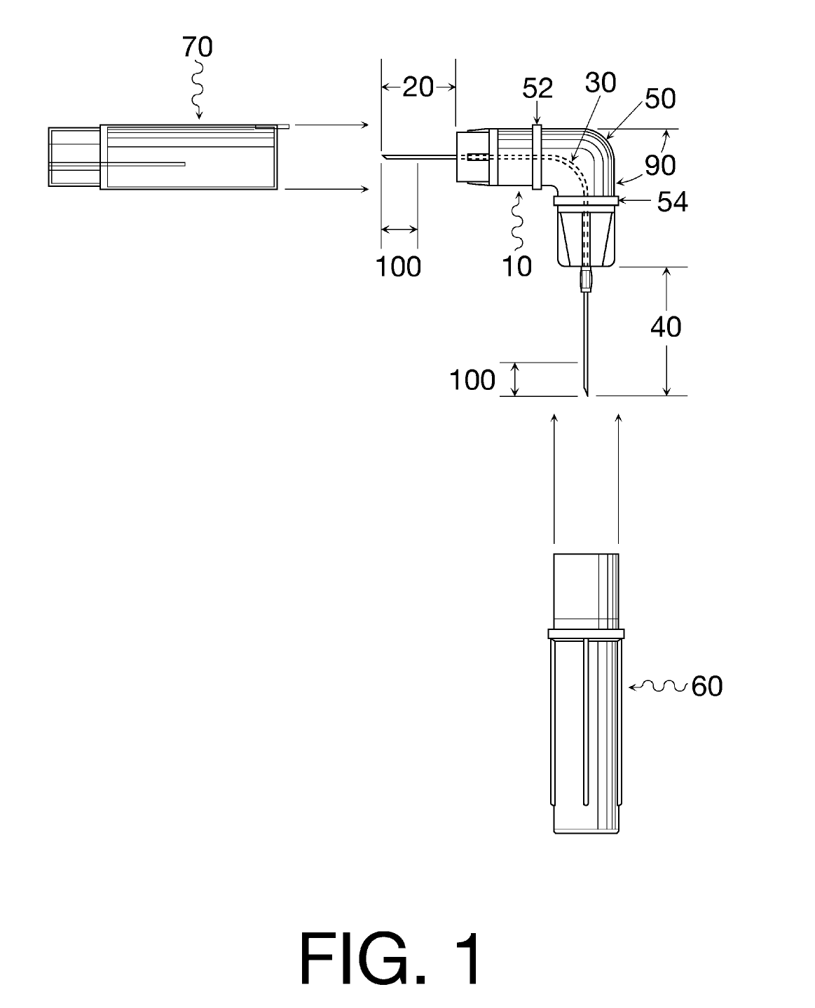

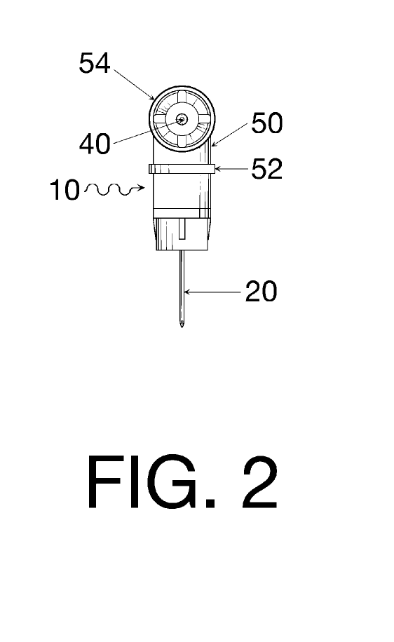

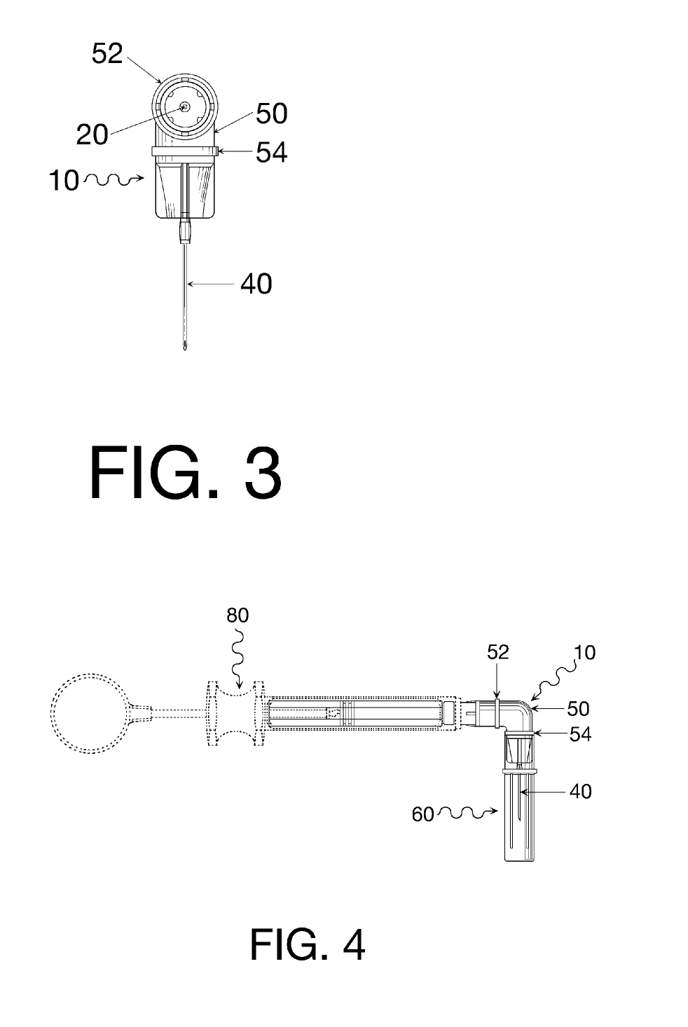

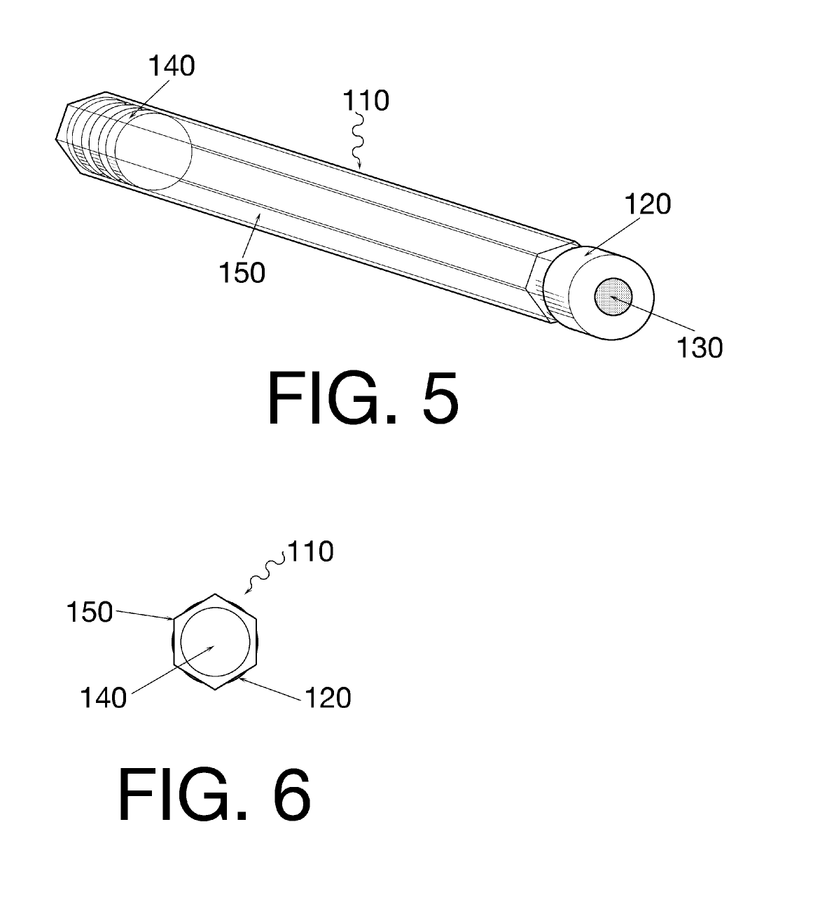

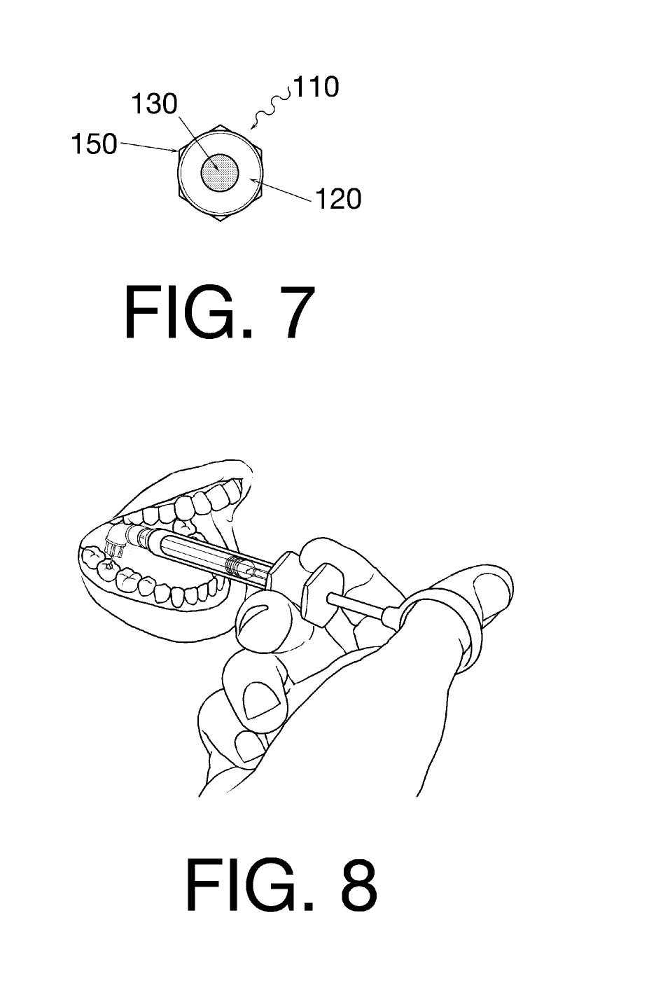

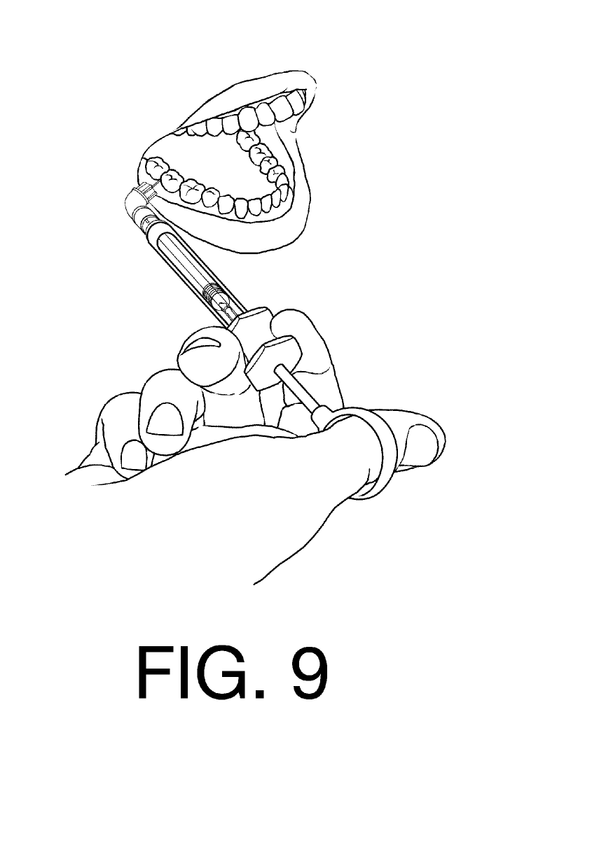

### DEFINITION LIST 1

| Term | Definition |
|---|---|
| 10 | Pre-angled needle assembly |
| 20 | Proximal portion of pre-angled needle assembly |
| 30 | Needle |
| 40 | Distal portion of pre-angled needle assembly |
| 50 | Hub of pre-angled needle assembly |
| 52 | Proximal end land area of pre-angled needle assembly |
| 54 | Distal end land area of pre-angled needle assembly |
| 60 | Distal safety cap of pre-angled needle assembly |
| 70 | Proximal safety cap of pre-angled needle assembly |
| 80 | Conventional anesthetic syringe assembly |
| 90 | Angle of pre-angled needle assembly |
| 100 | Needle tip of pre-angled needle assembly |
| 110 | Cartridge ampule with faceted distinguishability means |
| 120 | Cap of cartridge ampule |
| 130 | Flexible membrane of cartridge ampule |
| 140 | Piston of cartridge ampule |
| 150 | Tubular body of cartridge ampule |

### DETAILED DESCRIPTION OF INVENTION

The oral cavity liquid delivery system, depicted in FIG. 4, utilizes conventional anesthetic syringes 80 or conventional intraligamental syringes, novel cartridge ampules 110 filled with anesthetic or irrigation solution that are attachable to the syringe, and novel pre-angled needle assemblies 10 also attachable to the syringe 80.

The practitioner chooses from the system a cartridge ampule 110 containing the appropriate liquid for the procedure at hand and inserts the ampule into the syringe 80. The system allows for ampules containing various anesthetics and irrigation solutions.

Novel cartridge ampules 110 of the current invention contain solution other than anesthetic, phosphoric acid in certain concentrations, or hydrocolloid impression material. These cartridges will have a means to distinguish them (see claim 12) from other types of cartridges used for injection purposes already in the public domain. First is the faceted means where the cartridge has a smooth round inner bore, as with currently available cartridges, and a faceted exterior surface, i.e. hexagon, octagon, decagon, or other polygonal shape, with overall diameter equal to that of a standard cartridge to permit fitting the cartridge into a syringe. The faceted shape will alert the operator to the fact that the cartridge ampule does not contain anesthetic. Second is the decal means where the cartridge ampules are covered with an opaque boldly colored decal that makes the cartridges pertaining to this invention easily distinguishable from anesthetic cartridges. The label decal will have one or more longitudinal narrow slits that are transparent to allow the operator to determine how much liquid remains within the carpule. Third is the ink means where the cartridge includes areas applied with ink or paint that may contain bar codes and will contain signification that cartridge ampules with ink means are not intended for injection purposes. There is also a combination means where the cartridges include the faceted means, decal means and/or ink means to prevent confusion between novel cartridges pertaining to this invention and non-anesthetic cartridges already in the public domain. Other equivalent distinguishability means are also included with this invention. The best mode is the combination means.

After loading the appropriate cartridge ampule 110, the practitioner chooses from the system a pre-angled needle assembly 10 with the appropriate angle 90, needle tip 100, and length of distal portion of pre-angled needle assembly 40 for the procedure at hand and attaches the pre-angled needle assembly 10 to the syringe 80.

The syringe attachment means (see claim 3) may consist of: a threaded attachment means where a threaded coupling at the proximal end of the pre-angled needle assembly hub 50 matches the nipple threads on the distal end of a anesthetic syringe 80; a fin attachment means, which is the preferred embodiment, where several longitudinal flanges or fins (usually four) protrude radially inward from the inside of the proximal end of the pre-angled needle assembly hub 50 which engage the threads on the syringe nipple as the needle assembly is twisted onto the syringe 80 causing the fins to become threaded and mate with the syringe nipple as the needle assembly is twisted onto the syringe 80; or by other equivalent means including various adapter apparatus means.

After the pre-angled needle assembly 10 is attached to the syringe 80, the practitioner performs the specific oral cavity liquid delivery procedure or procedures and then re-caps the needle using the single-handed “scoop” technique or a needle re-capping device. With the current invention, the needle assembly is disposed of without directly touching the needle itself. Therefore, the needle is disposed of legally and safely. Additionally, the risk of needle puncture is greatly reduced.

In the case of irrigation procedures, the practitioner can quickly and easily change irrigation solutions by simply changing cartridge ampules 110. There is no complex and costly set-up as with the manual delivery or bag-and-tube systems. There is no risk of spilling. Most importantly, there is no risk of contaminating the patent's oral cavity with non-sterile irrigants.

What is claimed is:

1. A pre-angled needle assembly, comprising: a needle having an aperture running therethrough; a distal portion; a proximal portion; and an angled portion; wherein said needle is one continuous member throughout said distal, proximal, and angled portions of pre-angled needle assembly and said angled portion of pre-angled needle assembly joins said distal portion of pre-angled needle assembly to said proximal portion of pre-angled needle assembly at an angle.

2. A pre-angled needle assembly as in claim 1, wherein the length of said proximal portion of pre-angled needle assembly shall duplicate that of currently available anesthetic needles and is long enough to penetrate the membrane of a cartridge ampule yet not too long to interfere with the insertion and removal of cartridge ampules in a syringe.

3. A pre-angled needle assembly as in claim 2, wherein said angled portion of pre-angled needle assembly further comprises: a hub made of rigid material that fully encapsulates said needle running therethrough, wherein said hub further comprises a proximal end land area and a distal end land area; and a syringe attachment means located at the proximal end of said hub to reversibly connect said pre-angled needle assembly to a syringe.

4. A pre-angled needle assembly as in claim 3, further comprising: a proximal safety cap reversibly attached to the proximal end of said hub at said proximal end land area, wherein said proximal safety cap completely covers said proximal portion of pre-angled needle assembly when attached to said hub.

5. A pre-angled needle assembly as in claim 4, further comprising: a distal safety cap reversibly attached to the distal end of said hub at said distal end land area, wherein said distal safety cap completely covers said distal portion of pre-angled needle assembly when attached to said hub.

6. A pre-angled needle assembly as in claim 5, wherein the length of said distal portion of pre-angled needle assembly ranges from 5-30 millimeters.

7. A pre-angled needle assembly as in claim 6, wherein said angle, defined between the axis of said needle at said proximal portion of pre-angled needle assembly and the axis of said needle at said distal portion of pre-angled needle assembly, ranges from 5-25 or, alternatively, 45-175 degrees.

8. A pre-angled needle assembly as in claim 7, wherein said needle is made of sterile surgical stainless steel of the type used in currently available needles and ranges in size from 30-22 gauge.

9. A pre-angled needle assembly as in claim 8, wherein said hub is made of metal, plastic, ceramic, fiberglass, or any combination thereof.

10. A pre-angled needle assembly as in claim 9, wherein said needle further comprises: a needle tip located at the terminal end of said distal portion of pre-angled needle assembly, wherein said needle tip is: a sharp beveled penetrating needle tip to perform injections; a blunt-ended side port ejection irrigation tip; a notched-end side ejection tip used for irrigating; or a tip with a flat distal end that terminates at a ninety-degree angle to the axis of said needle at said distal portion of pre-angled needle assembly.

11. A plunger-type tubular cartridge ampule containing certain sterile fluid other than: 1) anesthetic, 2) phosphoric acid in 37% solution, 3) phosphoric acid in 50% solution, or 4) hydrocolloid impression material.

12. A plunger-type cartridge ampule as in claim 11, further comprising: a tubular body; a piston, wherein said piston is movable axially across the long axis of said tubular body and seals and contains said certain sterile fluid within said tubular body; a cap, wherein said cap further comprises a collar and flexible membrane both secured to said tubular body and seals and contains said certain sterile fluid within said tubular body; and a means to distinguish said cartridge ampule from other cartridge ampules already in the public domain containing anesthetic or other fluid intended for injection purposes.

13. A plunger-type cartridge ampule as in claim 12, wherein said certain sterile fluid is any one of the group consisting of sterile water, sterile saline solution, sodium hypochlorite, chloroform, EDTA solution, hydrogen peroxide, ethyl alcohol, eucalyptol, and chlorhexidine gluconate.

14. An oral cavity liquid delivery system, comprising: said pre-angled needle assembly; said cartridge ampule; and an anesthetic syringe.

15. An oral cavity liquid delivery system as in claim 14, wherein said anesthetic syringe further comprises: a body member having means providing a cavity to accommodate therewithin one of said cartridge ampules, and having axially disposed openings at the front and rear ends thereof; a plunger member extending through the opening at the rear end of said cavity and slidable with respect thereto, said plunger having a head at its forward end adapted to engage said movable piston member to slide the same forwardly and rearwardly within said tubular body, and a manually engageable actuator at the rear end thereof for pressing said plunger forward; and an adaptor within the opening at the front end of the syringe body member, said adapter being arranged to receive and removably secure said pre-angled needle assembly with axial alignment between said tubular body and said proximal portion of pre-angled needle assembly wherein the same extends through said flexible membrane into said certain sterile fluid.

16. An oral cavity liquid delivery system, comprising: said pre-angled needle assembly; said cartridge ampule; and an intraligamental syringe.

17. A method for performing an oral cavity injection using said pre-angled needle assembly, comprising the steps of: loading an anesthetic cartridge ampule into a syringe;
attaching said pre-angled needle assembly to the syringe; performing an oral cavity injection; recapping the distal portion of said pre-angled needle assembly with the distal safety cap of said pre-angled needle assembly; and removing said pre-angled needle assembly from the syringe.

18. A method for performing an oral cavity injection as defined by claim 17, wherein said step of attaching said pre-angled needle assembly to the syringe, comprises: choosing the appropriate pre-angled needle assembly from the oral cavity liquid delivery system with appropriate angle, appropriate length of distal portion of said pre-angled needle assembly, and appropriate needle tip of said pre-angled needle assembly to perform the specific oral cavity injection procedure at hand; and attaching said appropriate pre-angled needle assembly to a conventional anesthetic syringe.

19. A method for performing an oral cavity injection as defined by claim 17, wherein said step of performing an oral cavity injection comprises: inserting the needle tip of said pre-angled needle assembly into the pulp chamber; expressing anesthetic into pulp to accomplish an intra-pulpal injection; and removing said pre-angled needle assembly from the oral cavity.

20. A method for performing an oral cavity injection as defined by claim 17, wherein said step of performing an oral cavity injection comprises: inserting the needle tip of said pre-angled needle assembly into the periodontal ligament space; pressing and otherwise supporting said pre-angled needle assembly apically and towards the mesio-distal center of the tooth with fingers of the non-dominant hand positioned on said pre-angled needle assembly; expressing anesthetic to accomplish an intraligamental injection; and removing said pre-angled needle assembly from the oral cavity.

21. A method for performing an oral cavity injection as defined by claim 17, wherein said step of recapping the distal portion of said pre-angled needle assembly with the distal safety cap of said pre-angled needle assembly comprises: placing the proximal end of the distal safety cap of said pre-angled needle assembly onto the distal portion of said pre-angled needle assembly, by performing a single-handed “scoop” technique thereby scooping said distal safety cap of pre-angled needle assembly onto said distal portion of pre-angled needle assembly, or through the use of a needle re-capping device; and pressing said distal safety cap of pre-angled needle assembly onto the hub of said pre-angled needle assembly until seated on the distal end land area of said pre-angled needle assembly.

22. A method for performing an oral cavity irrigation using said pre-angled needle assembly, comprising the steps of: loading a plunger-type cartridge ampule into a syringe; attaching said pre-angled needle assembly to the syringe; performing an oral cavity irrigation; recapping the distal portion of said pre-angled needle assembly with the distal safety cap of said pre-angled needle assembly; and removing said pre-angled needle assembly from the syringe.

23. A method for performing an oral cavity irrigation as defined by claim 22, wherein said step of loading a plunger-type tubular cartridge ampule into the syringe comprises: choosing the appropriate plunger-type cartridge ampule from the oral cavity liquid delivery system with appropriate certain sterile fluid to perform the specific oral cavity irrigation procedure at hand; and loading said appropriate plunger-type cartridge ampule into a conventional syringe.

24. A method for performing an oral cavity irrigation as defined by claim 22, wherein said step of attaching said pre-angled needle assembly to the syringe, comprises: choosing the appropriate pre-angled needle assembly from the oral cavity liquid delivery system with appropriate angle, appropriate length of distal portion of said pre-angled needle assembly, and appropriate needle tip of said pre-angled needle assembly to perform the specific oral cavity irrigation procedure at hand; and attaching said appropriate pre-angled needle assembly to a conventional anesthetic syringe.

25. A method for performing an oral cavity irrigation as defined by claim 22, wherein said step of performing an oral cavity irrigation comprises: directing the needle tip of the distal portion of said pre-angled needle assembly towards the handpiece bur during tooth sectioning or bone removal procedures or into the extraction site after tooth removal has been accomplished; depressing the plunger of the syringe to force liquid out of the said needle tip of distal portion of pre-angled needle assembly into the operating site; and removing said pre-angled needle assembly from the oral cavity.

26. A method for performing an oral cavity irrigation as defined by claim 22, wherein said step of performing an oral cavity irrigation comprises: directing the needle tip of the distal portion of said pre-angled needle assembly into the pulp chamber or the coronal portion of a root canal preparation; depressing the plunger of the syringe to force liquid out of said needle tip of distal portion of pre-angled needle assembly into the root canal preparation area; and removing said pre-angled needle assembly from the oral cavity.

27. A method for performing an oral cavity irrigation as defined by claim 22, wherein said step of recapping the distal portion of said pre-angled needle assembly with the distal safety cap of said pre-angled needle assembly comprises: placing the proximal end of the distal safety cap of said pre-angled needle assembly onto the distal portion of said pre-angled needle assembly, by performing a single-handed “scoop” technique thereby scooping said distal safety cap of pre-angled needle assembly onto said distal portion of pre-angled needle assembly, or through the use of a needle re-capping device; and pressing said distal safety cap of pre-angled needle assembly onto the hub of said pre-angled needle assembly until seated on the distal end land area os said pre-angled needle assembly.

28. A method for performing oral cavity irrigation during restorative procedures using said pre-angled needle assembly, comprising the steps of: loading a plunger-type cartridge ampule into a syringe; attaching said pre-angled needle assembly to the syringe; performing an oral cavity irrigation; recapping the distal portion of said pre-angled needle assembly with the distal safety cap of said pre-angled needle assembly; and removing said pre-angled needle assembly from the syringe.

29. A method for performing oral cavity irrigation during restorative procedures as defined by claim 28, wherein said step of loading a plunger-type tubular cartridge ampule into the syringe comprises: choosing the appropriate plunger-type cartridge ampule from the oral cavity liquid delivery system with appropriate certain sterile fluid to perform the specific oral cavity irrigation procedure at hand; and loading said appropriate plunger-type cartridge ampule into a conventional syringe.

30. A method for performing oral cavity irrigation during restorative procedures as defined by claim 28, wherein said step of attaching said pre-angled needle assembly to the syringe, comprises: choosing the appropriate pre-angled needle assembly from the oral cavity liquid delivery system with appropriate angle, appropriate length of distal portion of said pre-angled needle assembly, and appropriate needle tip of said pre-angled needle assembly to perform the specific oral cavity irrigation procedure at hand; and attaching said appropriate pre-angled needle assembly to a conventional anesthetic syringe.

31. A method for performing oral cavity irrigation during restorative procedures as defined by claim 28, wherein said step performing an oral cavity irrigation, comprises: any of the conventional steps for performing tooth cavity irrigation, tooth cavity disinfection, or application of liquid drying agents to a tooth cavity, as well as post hole irrigation, post hole disinfection, or application of liquid drying agents to a post hole.

32. A method for performing oral cavity irrigation during restorative procedures as defined by claim 28, wherein said step of recapping the distal portion of said pre-angled needle assembly with the distal safety cap of said pre-angled needle assembly comprises: placing the proximal end of the distal safety cap of said pre-angled needle guidance assembly onto the distal portion of said pre-angled needle guidance assembly, by performing a single-handed “scoop” technique thereby scooping said distal safety cap of pre-angled needle assembly onto said distal portion of pre-angled needle assembly, or through the use of a needle re-capping device; and pressing said distal safety cap of pre-angled needle assembly onto the hub of said pre-angled needle assembly until seated on the distal end land area a said pre-angled needle assembly.
